(19) United States
(12) Patent Application Publication
(10) Pub. No.: US 2012/0071554 A1
(43) Pub. Date: Mar. 22, 2012

(54) DEUTERATED 2-PROPYLPENTANOIC ACID COMPOUNDS

(76) Inventor: Julie F. Liu, Lexington, MA (US)

(21) Appl. No.: 13/126,384

(22) PCT Filed: Oct. 28, 2009
(86) PCT No.: PCT/US09/62397

§ 371 Date: Dec. 12, 2011

Related U.S. Application Data
(60) Provisional application No. 61/109,046, filed on Oct. 28, 2008.

(51) Int. Cl. A61K 31/19; A61P 25/00; A61P 25/06; A61P 25/18; A61P 11/06; A61P 17/02; A61P 25/36; A61P 25/30; A61P 35/02; A61P 35/00; A61P 29/00; A61P 17/00; C07C 53/128; A61P 25/08

(52) U.S. Cl. 514/557; 562/606

(57) ABSTRACT

Described herein are novel analogs of 2-propylpentanoic acid, pharmaceutical compositions comprising the same, and methods of using the same for the treatment of diseases or conditions that are beneficially treated by administering a GABAAergic transmission enhancer and/or a histone deacetylase (HDAC) inhibitor.

### CROSS-REFERENCE TO PRIOR APPLICATIONS

This application claims priority to U.S. Provisional Application Ser. No. 61/109,046, filed Oct. 28, 2008, which is incorporated by reference herein in its entirety.

### TECHNICAL FIELD

This disclosure relates to novel analogs of 2-propylpentanoic acid and pharmaceutically acceptable salts thereof. This disclosure also provides compositions comprising a compound as provided herein and the use of such compositions in methods of treating diseases and conditions beneficially treated by administering a GABAAergic transmission enhancer and/or a histone deacetylase (HDAC) inhibitor.

### BACKGROUND

Many current medicines suffer from poor absorption, distribution, metabolism and/or excretion (ADME) properties that prevent their wider use. Poor ADME properties are also a major reason for the failure of drug candidates in clinical trials. While formulation technologies and prodrug strategies can be employed in some cases to improve certain ADME properties, these approaches have failed to overcome the inherent ADME problems that exist for many drugs and drug candidates. One inherent problem is the rapid metabolism that causes a number of drugs, which otherwise would be highly effective in treating a disease, to be cleared too rapidly from the body. A possible solution to rapid drug clearance is frequent or high dosing to attain a sufficiently high plasma level of drug. This, however, introduces a number of potential treatment problems, such as poor patient compliance with the dosing regimen, side effects that become more acute with higher doses, and increased cost of treatment.

In some select cases, a metabolic inhibitor will be co-administered with an important drug that is rapidly cleared. Such is the case with the protease inhibitor class of drugs that are used to treat HIV infection. These drugs are typically co-dosed with ritonavir, an inhibitor of cytochrome P450 enzyme CYP3A4, the enzyme responsible for their metabolism. Ritonavir itself has side effects and it adds to the pill burden for HIV patients who must already take a combination of different drugs. Similarly, dextromethorphan which undergoes rapid CYP2D6 metabolism is being tested in combination with the CYP2D6 inhibitor quinidine for the treatment of pseudobulbar disease.

In general, combining drugs with cytochrome P450 inhibitors is not a satisfactory strategy for decreasing drug clearance. The inhibition of a CYP enzyme activity can affect the metabolism and clearance of other drugs metabolized by that same enzyme. This can cause those other drugs to accumulate in the body to toxic levels.

A potentially attractive strategy, if it works, for improving a drug's metabolic properties is deuterium modification. In this approach, one attempts to slow the CYP-mediated metabolism of a drug by replacing one or more hydrogen atoms with deuterium atoms. Deuterium is a safe, stable, non-radioactive isotope of hydrogen. Deuterium forms stronger bonds with carbon than hydrogen does. In select cases, the increased bond strength imparted by deuterium can positively impact the ADME properties of a drug, creating the potential for improved drug efficacy, safety, and tolerability. At the same time, because the size and shape of deuterium are essentially identical to hydrogen, replacement of hydrogen by deuterium would not be expected to affect the biochemical potency and selectivity of the drug as compared to the original chemical entity that contains only hydrogen.

Over the past 35 years, the effects of deuterium substitution on the rate of metabolism have been reported for a very small percentage of approved drugs (see, e.g., Blake, M I et al, J Pharm Sci, 1975, 64:367-91; Foster, A B, Adv Drug Res 1985, 14:1-40 (“Foster”); Kushner, D J et al, Can J Physiol Pharmacol 1999, 79-88; Fisher, M B et al, Curr Opin Drug Discov Devel, 2006, 9:101-09 (“Fisher”)). The results have been variable and unpredictable. For some compounds deuteration caused decreased metabolic clearance in vivo. For others, there was no change in metabolism. Still others demonstrated decreased metabolic clearance. The variability in deuterium effects has also led experts to question or dismiss deuterium modification as a viable drug design strategy for inhibiting adverse metabolism. (See Foster at p. 35 and Fisher at p. 101).

The effects of deuterium modification on a drug's metabolic properties are not predictable even when deuterium atoms are incorporated at known sites of metabolism. Only by actually preparing and testing a deuterated drug can one determine if and how the rate of metabolism will differ from that of its undeuterated counterpart. Many drugs have multiple sites where metabolism is possible. The site(s) where deuterium substitution is required and the extent of deuteration necessary to see an effect on metabolism, if any, will be different for each drug.

Valproate, also known as sodium hydrogen bis(2-propylpentanoate), exerts its effect by a mechanism which has yet to be established. It has been suggested that its activity in epilepsy is related to increased brain concentrations of gamma-aminobutyric acid (GABA), reduced release and/or effects of excitatory amino acids, blockade of voltage-gated sodium channels and/or modulation of dopaminergic and serotoninergic transmission (see FDA label at www.fda.gov/cder/foi/label).

Valproate is currently approved for epilepsy, bipolar disorder, and migraine and in clinical trials for schizophrenia, autism, mood disorder, major depressive disorder, borderline personality disorder, intermittent explosive disorder, ADHD, dementia, Alzheimer's disease, amyotrophic lateral sclerosis, Huntington's disease, bronchial asthma, post-traumatic seizure disorder, spinal muscular atrophy, progressive supranuclear palsy (PSP), opiate dependence; drug dependence; substance withdrawal syndrome, nasopharyngeal carcinoma, acute myelogenous leukemia, myelodysplastic syndromes, lymphoma, advanced neoplasms, cervical cancer, breast cancer, malignant melanoma, Kaposi's sarcoma, lung cancer, and neurologic cancer.

Valproate is almost entirely metabolized by the liver. Adult patients on monotherapy excrete 30-50% of administered dose in the urine as the glucuronide conjugate, over 40% of the dose as the products of mitochondrial beta-oxidation, and less than 15-20% of the dose as products of other oxidative mechanisms. Less than 3% of the dose is excreted unchanged in the urine. In pediatric patients co-dosed with valproate and aspirin, inhibition of the beta-oxidation metabolic pathway lead to a 4-fold increase of the valproate free-fraction. (See FDA label @ http://www.fda.gov/cder/foi/label/2006/18081s44,18082s27,18723s33,19680s22,20593s15,2116 8s141b1.pdf).

Adverse events reported for patients treated with valproate include, but are not limited to, somnolence, dyspepsia, nausea, vomiting, diarrhea, anorexia, thrombocytopenia, dizziness, tremor, pain, abdominal pain, back pain, alopecia, weight gain, accidental injury, asthenia, infection, diplopia, tinnitus, ataxia, nystagmus, and pharyngitis. Warnings stated in the label include hepatotoxicity, pancreatitis, urea cycle disorders, somnolence in the elderly, thrombocytopenia, post-traumatic seizures, and usage in pregnancy. (See FDA label @ http://www.fda.gov/cder/foi/label/2006/18081s44,18082s27,18723s33,19680s22,20593s15,2116 8s141b1.pdf).

Despite the beneficial activities of valproate, there is a continuing need for new compounds to treat the aforementioned diseases and conditions.

### SUMMARY

In one embodiment, provided is a compound selected from the group consisting of:

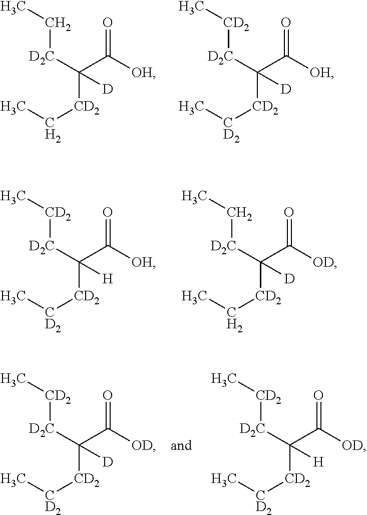

or a pharmaceutically acceptable salt thereof.

In another embodiment, provided is a pharmaceutically acceptable composition comprising a compound as described above or pharmaceutically acceptable salt thereof and a pharmaceutically acceptable carrier.

Also provided is a pharmaceutically acceptable composition comprising: (a) a compound of the Formula I:

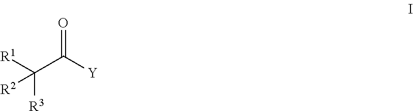

or a pharmaceutically acceptable salt thereof, where each of R¹ and R² independently for each occurrence is —CH₂CH₂CH₃, where —CH₂CH₂CH₃ is optionally substituted with 1 to 7 deuterium atoms; R³ is hydrogen or deuterium; and Y is OH or OD; provided that if each of R¹ and R² is —CH₂CH₂CH₃ or —CH₂—CHD-CH₂D, then R³ is deuterium; further provided that if R¹ is —CH₂CH₂CH₃ and R² is —CD₂CD₂CD₃; then R³ is deuterium; (b) a second therapeutic agent selected from the group consisting of sunitinib, sorafenib, dasatinib, erlotinib, lapatinib, lenalidomide, temozolomide, 5-fluorouracil, epirubicin, cyclophosphamide, karenitecin, decitabine, aripiprazole, olanzapine, lamotrigine, risperidone, quetiapine, carbamazepine, and phenyloin; and (c) a pharmaceutically acceptable carrier.

Also provided is a pharmaceutically acceptable composition comprising (a) a compound of formula:

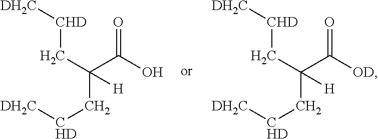

or a pharmaceutically acceptable salt thereof; (b) a second therapeutic agent selected from the group consisting of sunitinib, sorafenib, dasatinib, erlotinib, lapatinib, lenalidomide, temozolomide, 5-fluorouracil, epirubicin, cyclophosphamide, karenitecin, decitabine, aripiprazole, olanzapine, lamotrigine, risperidone, quetiapine, and phenyloin; and (c) a pharmaceutically acceptable carrier.

Also provided is a pharmaceutically acceptable composition comprising (a) a compound of formula:

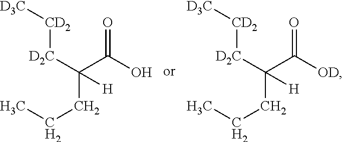

or a pharmaceutically acceptable salt thereof; (b) a second therapeutic agent selected from the group consisting of sunitinib, sorafenib, dasatinib, erlotinib, lapatinib, lenalidomide, temozolomide, 5-fluorouracil, epirubicin, cyclophosphamide, karenitecin, decitabine, aripiprazole, olanzapine, lamotrigine, risperidone, and quetiapine; and (c) a pharmaceutically acceptable carrier.

Also provided is a method of treating a patient suffering from or susceptible to a disease or condition selected from epilepsy, bipolar disorder, migraine, schizophrenia, autism, mood disorder, major depressive disorder, borderline personality disorder, intermittent explosive disorder, ADHD, dementia, Alzheimer's disease, amyotrophic lateral sclerosis, Huntington's disease, bronchial asthma, post-traumatic seizure disorder, spinal muscular atrophy, progressive supranuclear palsy (PSP), opiate dependence, drug dependence, substance withdrawal syndrome, nasopharyngeal carcinoma, acute myelogenous leukemia, myelodysplastic syndromes, lymphoma, advanced neoplasms, cervical cancer, breast cancer, malignant melanoma, Kaposi's sarcoma, lung cancer, neurologic cancer, basal cell carcinoma, squamous cell carcinoma, keratoacanthoma, Bowen disease, cutaneous T-cell lymphoma, non-small cell lung cancer, pre-malignant lesions, inflammations of the skin and/or mucosa, exposure to UV light, and sunburn, comprising the step of administering to the patient in need thereof a pharmaceutically acceptable composition comprising a compound of the Formula I:

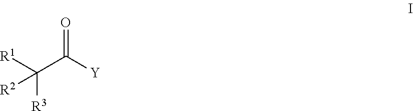

or a pharmaceutically acceptable salt thereof, where each of R¹ and R² independently for each occurrence is —CH₂CH₂CH₃, where —CH₂CH₂CH₃ is optionally substituted with 1 to 7 deuterium atoms; R³ is hydrogen or deuterium; and Y is OH or OD; provided that if each of R¹ and R² is —CH₂CH₂CH₃ or —CH₂CHD-CH₂D, then R³ is deuterium; further provided that if R¹ is —CH₂CH₂CH₃ and R² is —CD₂CD₂CD₃, then R³ is deuterium; and a pharmaceutically acceptable carrier.

Certain embodiments relate to the aforementioned method, where the patient is suffering from or susceptible to a disease or condition selected from epilepsy, bipolar disorder, migraine, schizophrenia, autism, mood disorder, major depressive disorder, borderline personality disorder, intermittent explosive disorder, ADHD, dementia, Alzheimer's disease, amyotrophic lateral sclerosis, Huntington's disease, bronchial asthma, post-traumatic seizure disorder, spinal muscular atrophy, progressive supranuclear palsy (PSP), opiate dependence, drug dependence, substance withdrawal syndrome, nasopharyngeal carcinoma, acute myelogenous leukemia, myelodysplastic syndromes, lymphoma, advanced neoplasms, cervical cancer, breast cancer, malignant melanoma, Kaposi's sarcoma, lung cancer, and neurologic cancer.

Certain embodiments relate to any of the aforementioned methods, where the patient is suffering from or susceptible to a disease or condition selected from epilepsy, bipolar disorder, and migraine.

Certain embodiments relate to any of the aforementioned methods, where the compound is selected from the group consisting of:

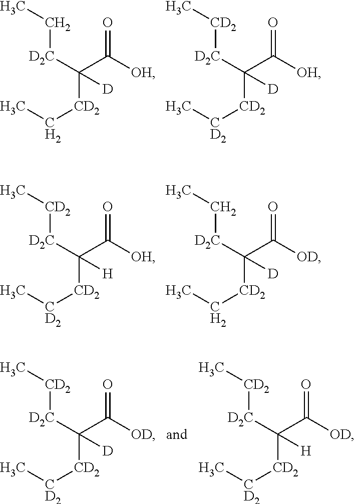

or a pharmaceutically acceptable salt thereof.

Also provided is a method of treating a patient suffering from or susceptible to a disease or condition selected from the group consisting of bipolar disorder, migraine, schizophrenia, autism, mood disorder, major depressive disorder, borderline personality disorder, intermittent explosive disorder, ADHD, dementia, Alzheimer's disease, amyotrophic lateral sclerosis, Huntington's disease, bronchial asthma, post-traumatic seizure disorder, spinal muscular atrophy, progressive supranuclear palsy (PSP), opiate dependence, drug dependence, substance withdrawal syndrome, nasopharyngeal carcinoma, acute myelogenous leukemia, myelodysplastic syndromes, lymphoma, advanced neoplasms, cervical cancer, breast cancer, malignant melanoma, Kaposi's sarcoma, lung cancer, neurologic cancer, basal cell carcinoma, squamous cell carcinoma, keratoacanthoma, Bowen disease, cutaneous T-cell lymphoma, non-small cell lung cancer, pre-malignant lesions, inflammations of the skin and/or mucosa, exposure to UV light, and sunburn, comprising the step of administering to the patient in need thereof a pharmaceutically acceptable composition comprising a compound selected from the group consisting of

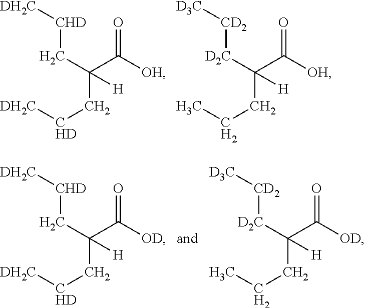

or pharmaceutically acceptable salts thereof and a pharmaceutically acceptable carrier.

Certain embodiments relate to the aforementioned method, where the patient is suffering from or susceptible to a disease or condition selected from the group consisting of bipolar disorder, migraine, schizophrenia, autism, mood disorder, major depressive disorder, borderline personality disorder, intermittent explosive disorder, ADHD, dementia, Alzheimer's disease, amyotrophic lateral sclerosis, Huntington's disease, bronchial asthma, post-traumatic seizure disorder, spinal muscular atrophy, progressive supranuclear palsy (PSP), opiate dependence, drug dependence, substance withdrawal syndrome, nasopharyngeal carcinoma, acute myelogenous leukemia, myelodysplastic syndromes, lymphoma, advanced neoplasms, cervical cancer, breast cancer, malignant melanoma, Kaposi's sarcoma, lung cancer, and neurologic cancer.

Certain embodiments relate to the aforementioned method, where the patient is suffering from or susceptible to a disease or condition selected from bipolar disorder and migraine.

Certain embodiments relate to any of the aforementioned methods, further comprising co-administering to the patient in need thereof a second therapeutic agent selected from one of more of: sunitinib, sorafenib, dasatinib, erlotinib, lapatinib, lenalidomide, temozolomide, 5-fluorouracil, epirubicin, cyclophosphamide, karenitecin, and decitabine, wherein the patient is suffering from or susceptible to cancer; a second therapeutic agent selected from one of more of: aripiprazole, olanzapine, and lamotrigine, wherein the patient is suffering from or susceptible to bipolar disorder; or a second therapeutic agent selected from one or more of risperidone, quetiapine, and olanzapine, wherein the patient is suffering from dementia.

Certain embodiments relate to any of the aforementioned methods, further comprising co-administering to the patient in need thereof a second therapeutic agent selected from one or more of carbamazepine and phenyloin, wherein the patient is suffering from epilepsy.

### DETAILED DESCRIPTION

As disclosed in greater detail below, provided herein are novel analogs of 2-propylpentanoic acid and pharmaceutically acceptable salts thereof, which are useful in the treatment of various diseases or conditions, e.g., in the treatment of disease states or conditions mediated, at least in part, by GABA, HDAC, or dopaminergic and/or serotoninergic transmission.

### DEFINITIONS

The terms “ameliorate” and “treat” are used interchangeably and include both therapeutic and prophylactic treatment. Both terms mean decrease, suppress, attenuate, diminish, arrest, or stabilize the development or progression of a disease (e.g., a disease or disorder delineated herein), lessen the severity of the disease or improve the symptoms associated with the disease.

“D” refers to deuterium.

“Disease” means any condition or disorder that damages or interferes with the normal function of a cell, tissue, or organ.

It will be recognized that some variation of natural isotopic abundance occurs in a synthesized compound depending upon the origin of chemical materials used in the synthesis.

Thus, a preparation of a 2-propylpentanoic analog will inherently contain small amounts of deuterated isotopologues. The concentration of naturally abundant stable hydrogen and carbon isotopes, notwithstanding this variation, is small and immaterial as compared to the degree of stable isotopic substitution of compounds provided herein. See, for instance, Wada E et al., Seikagaku 1994, 66:15; Gannes L Z et al., Comp Biochem Physiol Mol Integr Physiol 1998, 119:725.

The term “isotopic enrichment factor” as used herein means the ratio between the isotopic abundance and the natural abundance of a specified isotope.

In other embodiments, a compound of this disclosure has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

The term “isotopologue” refers to a species that has the same chemical structure and formula as a specific compound of this disclosure, with the exception of the positions of isotopic substitution and/or level of isotopic enrichment at one or more positions, e.g., H vs. D.

The term “compound,” when referring to a compound of this disclosure, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this disclosure will depend upon a number of factors including the isotopic purity of deuterated reagents used to make the compound and the efficiency of incorporation of deuterium in the various synthesis steps used to prepare the compound. However, as set forth above the relative amount of such isotopologues will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

A salt of a compound of this disclosure is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this disclosure. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

The pharmaceutically acceptable salt may also be a salt of a compound of the present invention having an acidic functional group, such as a carboxylic acid functional group, and a base. Exemplary bases include, but are not limited to, hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH-(C.sub.1-C.sub.6)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

The compounds of the present disclosure (e.g., compounds of Formula I, compound 100, compound 100a, compound 101, compound 101a, compound 102, compound 102a, and other 2-propylpentanoic acid analogs as described herein), may contain an asymmetric carbon atom, for example, as the result of deuterium substitution or otherwise. As such, compounds of this disclosure can exist as either individual enantiomers, or mixtures of the two enantiomers. Accordingly, a compound of the present disclosure may exist as either a racemic mixture or a scalemic mixture, or as individual respective stereoisomers that are substantially free from another possible stereoisomer.

The term “substantially free of other stereoisomers” as used herein means less than 25% of other stereoisomers, preferably less than 10% of other stereoisomers, more preferably less than 5% of other stereoisomers and most preferably less than 2% of other stereoisomers, are present. Methods of obtaining or synthesizing an individual enantiomer for a given compound are known in the art and may be applied as practicable to final compounds or to starting material or intermediates.

Unless otherwise indicated when a disclosed compound is named or depicted by a structure without specifying the stereochemistry and has one or more chiral centers, it is understood to represent all possible stereoisomers of the compound.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” refers to deuterium. “Stereoisomer” refers to both enantiomers and diastereomers. “Tert”, “^{t}”, and “t-” each refer to tertiary. “US” refers to the United States of America.

The term “substituted with deuterium atoms” means that one or more hydrogen atoms in the indicated moiety are substituted with a deuterium atom.

Throughout this specification, a variable may be referred to generally (e.g., “each R”) or may be referred to specifically (e.g., R¹, R², R³, etc.). Unless otherwise indicated, when a variable is referred to generally, it is meant to include all specific embodiments of that particular variable.

### Therapeutic Compounds

In one embodiment, provided is a compound selected from the group consisting of:

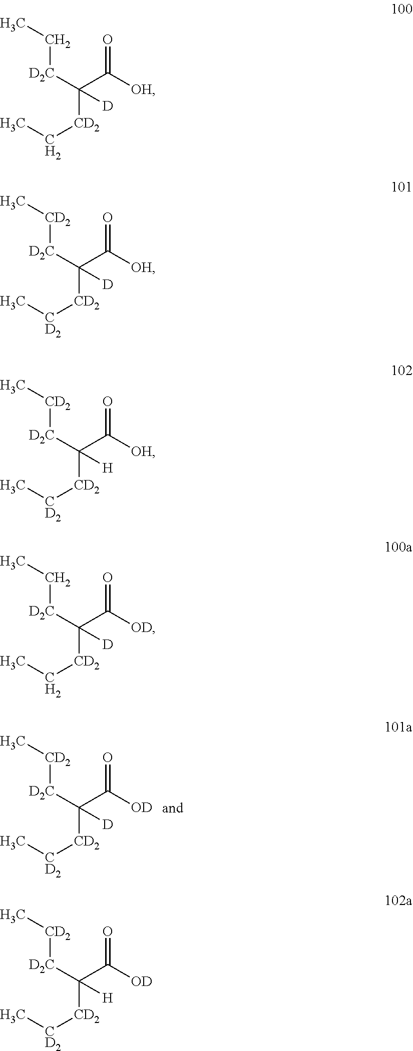

or a pharmaceutically acceptable salt thereof.

In another set of embodiments, any atom not designated as hydrogen or deuterium in any of the embodiments set forth herein is present at its natural isotopic abundance.

In another set of embodiments, the compounds of Formula 100, 100a, 101, 101a, 102, and 102a are provided in isolated form, e.g., the compound is not in a cell or organism and the compound is separated from some or all of the components that typically accompany it in nature.

The synthesis of compounds 100, 100a, 101, 101a, 102, and 102a, and other deuterated analogs of 2-propylpentanoic acid as described herein can be readily achieved by synthetic chemists of ordinary skill, for example by reference to the schemes shown herein. Relevant procedures and intermediates are disclosed, for instance in U.S. Pat. No. 4,155,929 and Great Britain patents GB1529786 and GB1522450.

Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure. Certain intermediates can be used with or without purification (e.g., filtration, distillation, sublimation, crystallization, trituration, solid phase extraction, and chromatography).

### Exemplary Synthesis

Compounds described herein may be prepared by reference to the known methods for making valproate. Certain intermediates or reagents useful for making valproate may be replaced with corresponding deuterated intermediates or reagents as may be needed depending on the desired site or sites of deuterium incorporation, as illustrated below.

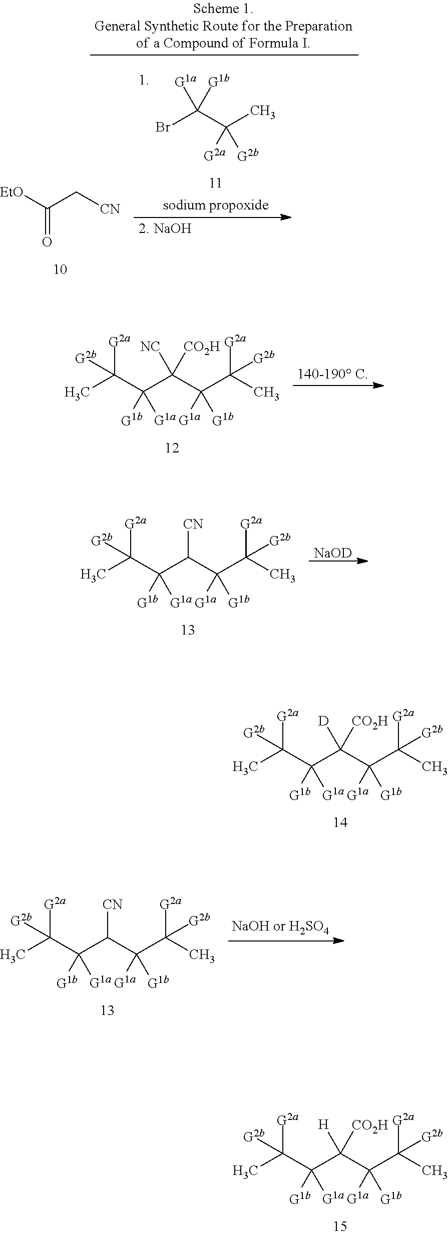

Scheme 1 shows a general synthetic route useful for preparing compounds 100, 101 and 102 as well as other deuterated versions of 2-propylpentanoic acid. In this scheme, each G is independently selected from H or D. It would be apparent to one skilled in the art that the terminal methyl groups in compound II may be replaced by CH₂D, CHD₂ or CD₃ to obtain other compounds of this disclosure.

Commercially available deuterated bromide reagents useful as reagent 11 include those listed below.

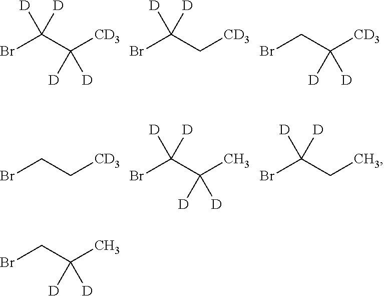

Compound 100 is synthesized using

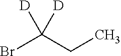

as reagent 11. Compounds 101 and 102 are synthesized using

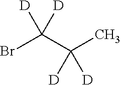

as reagent 11.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art. Additional methods of synthesizing the 2-propylpentanoic acid analogs as described herein and their synthetic precursors, including those within routes not explicitly shown in schemes herein, are within the means of chemists of ordinary skill in the art. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene T W et al., Protective Groups in Organic Synthesis, 3^{rd} Ed., John Wiley and Sons (1999); Fieser L et al., Fieser and Fieser's Reagents for Organic Synthesis, John Wiley and Sons (1994); and Paquette L, ed., Encyclopedia of Reagents for Organic Synthesis, John Wiley and Sons (1995) and subsequent editions thereof.

Combinations of substituents and variables envisioned by this invention are only those that result in the formation of stable compounds.

### Compositions

Also provided are pharmaceutical compositions comprising an effective amount of a compound of Formula 100, 100a, 101, 101a, 102, or 102a, or a pharmaceutically acceptable salt thereof; and a pharmaceutically acceptable carrier. In certain instances the pharmaceutical composition is pyrogen-free. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present disclosure in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this disclosure optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the present disclosure include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. Remington: The Science and Practice of Pharmacy, Lippincott Williams & Wilkins, Baltimore, Md. (20th ed. 2000).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present disclosure suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this disclosure may be administered in the form of suppositories for rectal administration. These compositions can be prepared by mixing a compound of this disclosure with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this disclosure may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this disclosure is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this disclosure include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this disclosure may also be topically applied to the lower intestinal tract by rectal suppository formulation or in a suitable enema formulation. Topically-transdermal patches and iontophoretic administration are also included in this disclosure.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this disclosure may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, provided is a method of coating an implantable medical device comprising the step of contacting said device with the coating composition described above. It will be obvious to those skilled in the art that the coating of the device will occur prior to implantation into a mammal.

According to another embodiment, provided is a method of impregnating an implantable drug release device comprising the step of contacting said drug release device with a compound or composition of this disclosure. Implantable drug release devices include, but are not limited to, biodegradable polymer capsules or bullets, non-degradable, diffusible polymer capsules and biodegradable polymer wafers.

According to another embodiment, provided is an implantable medical device coated with a compound or a composition comprising a compound of this disclosure, such that said compound is therapeutically active.

According to another embodiment, provided is an implantable drug release device impregnated with or containing a compound or a composition comprising a compound of this disclosure, such that said compound is released from said device and is therapeutically active.

Where an organ or tissue is accessible because of removal from the patient, such organ or tissue may be bathed in a medium containing a composition of this disclosure, a composition of this disclosure may be painted onto the organ, or a composition of this disclosure may be applied in any other convenient way.

Also provided is a composition comprising:

a) a compound of the Formula I:

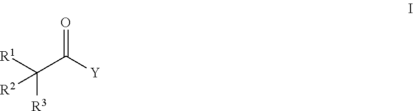

or a pharmaceutically acceptable salt thereof, where
each of R¹ and R² independently for each occurrence is —CH₂CH₂CH₃, wherein —CH₂CH₂CH₃ is optionally substituted with 1 to 7 deuterium atoms;
R³ is hydrogen or deuterium; and
Y is OH or OD,
   provided that if each of R¹ and R² is —CH₂CH₂CH₃ or —CH₂—CHD-CH₂D, then R³ is deuterium; further provided that if R¹ is —CH₂CH₂CH₃ and R² is —CD₂CD₂CD₃, then R³ is deuterium;

b) a pharmaceutically acceptable carrier; and

c) a second therapeutic agent.

In one embodiment, any atom not designated as hydrogen or deuterium in Formula I is present at its natural isotopic abundance.

One embodiment relates to the aforementioned composition, where the compound of Formula I is selected from the group consisting of compound 100, compound 100a, compound 101, compound 101a, compound 102 and compound 102a.

The second therapeutic agent may be selected from any compound or therapeutic agent known to have or that demonstrates advantageous properties when administered with a compound having the same mechanism of action as valproate. Such agents include those indicated as being useful in combination with valproate, including but not limited to, those described in WO 2003066039, WO 2005000289, WO 2005097138, WO 2006081347, and WO 2007054727.

In certain instances, the second therapeutic agent is an agent useful in the treatment or prevention of a disease or condition selected from epilepsy, bipolar disorder, migraine, schizophrenia, autism, mood disorder, major depressive disorder, borderline personality disorder, intermittent explosive disorder, ADHD, dementia, Alzheimer's disease, amyotrophic lateral sclerosis, Huntington's disease, bronchial asthma, post-traumatic seizure disorder, spinal muscular atrophy, progressive supranuclear palsy (PSP), opiate dependence, drug dependence, substance withdrawal syndrome, nasopharyngeal carcinoma, acute myelogenous leukemia, myelodysplastic syndromes, lymphoma, advanced neoplasms, cervical cancer, breast cancer, malignant melanoma, Kaposi's sarcoma, lung cancer, neurologic cancer, basal cell carcinoma, squamous cell carcinoma, keratoacanthoma, Bowen disease, cutaneous T-cell lymphoma, non-small cell lung cancer, pre-malignant lesions, inflammations of the skin and/or mucosa, protection from UV light, and treatment of sunburn.

In one embodiment, the second therapeutic agent is selected from sunitinib, sorafenib, dasatinib, erlotinib, lapatinib, lenalidomide, temozolomide, 5-fluorouracil, epirubicin, cyclophosphamide, karenitecin, decitabine, aripiprazole, olanzapine, lamotrigine, risperidone, quetiapine, carbamazepine, and phenyloin.

In another embodiment, provided is a composition comprising:

a) a compound of the formula

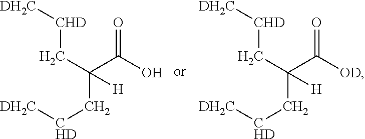

or a pharmaceutically acceptable salt thereof;

b) a pharmaceutically acceptable carrier; and

c) a second therapeutic agent selected from the second therapeutic agent is selected from sunitinib, sorafenib, dasatinib, erlotinib, lapatinib, lenalidomide, temozolomide, 5-fluorouracil, epirubicin, cyclophosphamide, karenitecin, decitabine, aripiprazole, olanzapine, lamotrigine, risperidone, quetiapine, and phenyloin.

In another embodiment, provided is a composition comprising:

a) a compound of the formula:

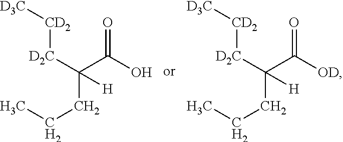

or a pharmaceutically acceptable salt thereof;

b) a pharmaceutically acceptable carrier; and

c) a second therapeutic agent selected from the second therapeutic agent is selected from sunitinib, sorafenib, dasatinib, erlotinib, lapatinib, lenalidomide, temozolomide, 5-fluorouracil, epirubicin, cyclophosphamide, karenitecin, decitabine, aripiprazole, olanzapine, lamotrigine, risperidone, and quetiapine.

In another set of embodiments, the compositions as described above are provided in isolated form, e.g., the compositions are not in a cell or organism.

In another embodiment, provided are separate dosage forms of an analog of 2-propylpentanoic acid compound as described herein, or a pharmaceutical salt thereof; and one or more of any of the corresponding, above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the present disclosure, the 2-propylpentanoic acid analog as described herein is present in an effective amount. As used herein, the term “effective amount” refers to an amount which, when administered in a proper dosing regimen, is sufficient to treat (therapeutically or prophylactically) the target disorder. For example, to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., (1966) Cancer Chemother. Rep 50: 219. Body surface area may be approximately determined from height and weight of the patient. See, e.g., Scientific Tables, Geigy Pharmaceuticals, Ardsley, N.Y., 1970, 537.

In one embodiment, an effective amount of a compound of this disclosure can range from about 0.1 mg/kg/day to about 600 mg/kg/day; from about 1 mg/kg/day to about 300 mg/kg/day, or from about 2 mg/kg/day to about 120 mg/kg/day, or from about 10 mg/kg/day to about 60 mg/kg/day. In certain embodiments, treatment is administered once daily.

Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician. For example, guidance for selecting an effective dose can be determined by reference to the prescribing information for valproate.

For pharmaceutical compositions that comprise a second therapeutic agent, an effective amount of the second therapeutic agent is between about 20% and 100% of the dosage normally utilized in a monotherapy regime using just that agent. in certain instances, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this disclosure. When this occurs, it will allow the effective dosage of the second therapeutic agent and/or the compound of this disclosure to be reduced from that required in a monotherapy. This has the advantage of minimizing toxic side effects of either the second therapeutic agent of a compound of this disclosure, synergistic improvements in efficacy, improved ease of administration or use and/or reduced overall expense of compound preparation or formulation.

### Methods of Treatment

According to another embodiment, provided is a method of treating a disease that is beneficially treated by valproate comprising the step of administering to a patient in need thereof a composition comprising:

a) a compound of the Formula I:

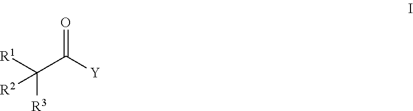

or a pharmaceutically acceptable salt thereof, where:
each of R¹ and R² independently for each occurrence is —CH₂CH₂CH₃, where —CH₂CH₂CH₃ is optionally substituted with 1 to 7 deuterium atoms;
R³ is hydrogen or deuterium; and
Y is OH or OD,
   provided that if each of R¹ and R² is —CH₂CH₂CH₃ or —CH₂—CHD-CH₂D, then R³ is deuterium; further provided that if R¹ is —CH₂CH₂CH₃ and R² is —CD₂CD₂CD₃; then R³ is deuterium; and

b) a pharmaceutically acceptable carrier.

Certain embodiments relate to the aforementioned method, where the disease that is beneficially treated by valproate is epilepsy, bipolar disorder, schizophrenia, autism, mood disorder, major depressive disorder, borderline personality disorder, intermittent explosive disorder, ADHD, dementia, Alzheimer's disease, amyotrophic lateral sclerosis, Huntington's disease, bronchial asthma, post-traumatic seizure disorder, spinal muscular atrophy, progressive supranuclear palsy (PSP), opiate dependence; drug dependence; substance withdrawal syndrome, nasopharyngeal carcinoma, acute myelogenous leukemia, myelodysplastic syndromes, lymphoma, advanced neoplasms, cervical cancer, breast cancer, malignant melanoma, Kaposi's sarcoma, lung cancer, or neurologic cancer.

One embodiment relates to the aforementioned method, where any atom not designated as deuterium in the compound of Formula I is present at its natural isotopic abundance.

Another embodiment relates to the aforementioned method, where the compound of Formula I is compound 100, compound 100a, compound 101, compound 101a, compound 102, or compound 102a.

Such diseases are well known in the art and are disclosed in, but not limited to the following patents and published applications: WO 1994027587, WO 2000066110, WO 2003066039, WO 2003103635, WO 2005000289, WO 2005123097, and WO 2007054727. Such diseases include, but are not limited to, epilepsy, bipolar disorder, migraine, schizophrenia, autism, mood disorder, major depressive disorder, borderline personality disorder, intermittent explosive disorder, ADHD, dementia, Alzheimer's disease, amyotrophic lateral sclerosis, Huntington's disease, bronchial asthma, post-traumatic seizure disorder, spinal muscular atrophy, progressive supranuclear palsy (PSP), opiate dependence; drug dependence; substance withdrawal syndrome, nasopharyngeal carcinoma, acute myelogenous leukemia, myelodysplastic syndromes, lymphoma, advanced neoplasms, cervical cancer, breast cancer, malignant melanoma, Kaposi's sarcoma, lung cancer, neurologic cancer, basal cell carcinoma, squamous cell carcinoma, keratoacanthoma, Bowen disease, cutaneous T-cell lymphoma, non-small cell lung cancer, pre-malignant lesions, inflammations of the skin and/or mucosa, protection from UV light, and treatment of sunburn.

In one embodiment, the method provided herein is used to treat a disease or condition selected from epilepsy, bipolar disorder, migraine, schizophrenia, autism, mood disorder, major depressive disorder, borderline personality disorder, intermittent explosive disorder, ADHD, dementia, Alzheimer's disease, amyotrophic lateral sclerosis, Huntington's disease, bronchial asthma, post-traumatic seizure disorder, spinal muscular atrophy, progressive supranuclear palsy (PSP), opiate dependence; drug dependence; substance withdrawal syndrome, nasopharyngeal carcinoma, acute myelogenous leukemia, myelodysplastic syndromes, lymphoma, advanced neoplasms, cervical cancer, breast cancer, malignant melanoma, Kaposi's sarcoma, lung cancer, and neurologic cancer in a patient in need thereof.

In another embodiment, the method provided herein is used to treat a disease or condition selected from epilepsy, bipolar disorder, and migraine in a patient in need thereof.

In another embodiment, provided is a method of treating a disease or condition selected from the group consisting of bipolar disorder, migraine, schizophrenia, autism, mood disorder, major depressive disorder, borderline personality disorder, intermittent explosive disorder, ADHD, dementia, Alzheimer's disease, amyotrophic lateral sclerosis, Huntington's disease, bronchial asthma, post-traumatic seizure disorder, spinal muscular atrophy, progressive supranuclear palsy (PSP), opiate dependence; drug dependence; substance withdrawal syndrome, nasopharyngeal carcinoma, acute myelogenous leukemia, myelodysplastic syndromes, lymphoma, advanced neoplasms, cervical cancer, breast cancer, malignant melanoma, Kaposi's sarcoma, lung cancer, neurologic cancer, basal cell carcinoma, squamous cell carcinoma, keratoacanthoma, Bowen disease, cutaneous T-cell lymphoma, non-small cell lung cancer, pre-malignant lesions, inflammations of the skin and/or mucosa, exposure to UV light, and sunburn in a patient in need thereof, comprising the step of administering to the patient a pharmaceutically acceptable composition comprising:

a) a compound selected from the group consisting of

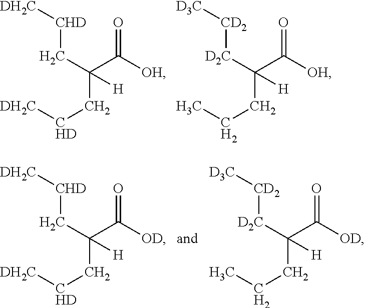

or a pharmaceutically acceptable salt thereof; and

b) a pharmaceutically acceptable carrier.

Certain embodiments relate to the aforementioned method, where the disease or condition is selected from the group consisting of bipolar disorder, migraine, schizophrenia, autism, mood disorder, major depressive disorder, borderline personality disorder, intermittent explosive disorder, ADHD, dementia, Alzheimer's disease, amyotrophic lateral sclerosis, Huntington's disease, bronchial asthma, post-traumatic seizure disorder, spinal muscular atrophy, progressive supranuclear palsy (PSP), opiate dependence; drug dependence, substance withdrawal syndrome, nasopharyngeal carcinoma, acute myelogenous leukemia, myelodysplastic syndromes, lymphoma, advanced neoplasms, cervical cancer, breast cancer, malignant melanoma, Kaposi's sarcoma, lung cancer, and neurologic cancer.

In certain instances, the disease or condition can be bipolar disorder or migraine.

Identifying a patient in need of such treatment can be in the judgment of a patient or a health care professional and can be subjective (e.g. opinion) or objective (e.g. measurable by a test or diagnostic method).

In another embodiment relates to any of the aforementioned methods, further comprising the step of co-administering to the patient in need thereof one or more second therapeutic agents. The choice of second therapeutic agent may be made from any second therapeutic agent known to be useful for co-administration with valproate. The choice of second therapeutic agent is also dependent upon the particular disease or condition to be treated. Examples of second therapeutic agents that may be employed in the methods of this disclosure are, for example, analogs as set forth above for use in combination compositions comprising a 2-propylpentanoic acid as described herein and a second therapeutic agent.

In certain embodiments, the combination therapies provided herein comprise the step of co-administering to the patient in need thereof (a) a compound selected from the group consisting of:

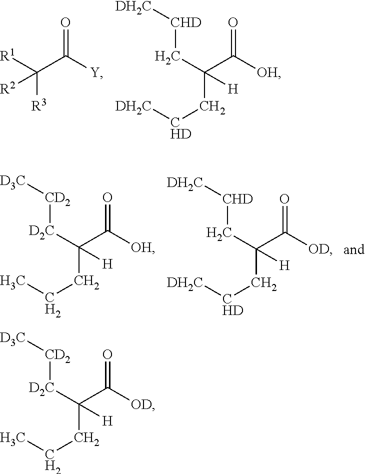

or a pharmaceutically acceptable salt thereof, where R¹, R², R³ and Y are as defined above; and (b) a second therapeutic agent (indicated in parentheses) for treatment of the following conditions: cancer (sunitinib, sorafenib, dasatinib, erlotinib, lapatinib, lenalidomide, temozolomide, 5-fluorouracil, epirubicin, cyclophosphamide, karenitecin, and/or decitabine); bipolar disorder (aripiprazole, olanzapine, and/or lamotrigine); and dementia (risperidone, quetiapine, and/or olanzapine). Another embodiment relates to the aforementioned method, where the compound is compound 100, compound 100a, compound 101, compound 101a, compound 102, or compound 102a.

In another embodiment, the combination therapies of this disclosure include co-administering to the patient in need thereof a compound of the Formula I:

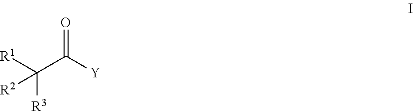

or a pharmaceutically acceptable salt thereof, R¹, R², R³ and Y are as defined above; and carbamazepine and/or phenyloin for treatment of epilepsy. Another embodiment relates to the aforementioned method, where the compound is compound 100, compound 100a, compound 101, compound 101a, compound 102, or compound 102a.

In another set of embodiments, the 2-propylpentanoic acid analogs used in any of the methods described above are provided in isolated form, e.g., the compound is not in a cell or organism and the compound is separated from some or all of the components that typically accompany it in nature.

The term “co-administered” as used herein means that the second therapeutic agent may be administered together with a compound of this disclosure as part of a single dosage form (such as a composition as described herein comprising a compound of this disclosure and an second therapeutic agent as described above) or as separate, multiple dosage forms. Alternatively, the additional agent may be administered prior to, consecutively with, or following the administration of a compound of this disclosure. In such combination therapy treatment, both the compounds of this disclosure and the second therapeutic agent(s) are administered by conventional methods. The administration of a composition of this disclosure, comprising both a 2-propylpentanoic acid as described herein and a second therapeutic agent, to a patient does not preclude the separate administration of that same therapeutic agent, any other second therapeutic agent or any compound of this disclosure to said patient at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of this disclosure, where a second therapeutic agent is administered to a subject, the effective amount of the compound of this disclosure is less than its effective amount would be where the second therapeutic agent is not administered. In another embodiment, the effective amount of the second therapeutic agent is less than its effective amount would be where the compound of this disclosure is not administered. In this way, undesired side effects associated with high doses of either agent may be minimized. Other potential advantages (including without limitation improved dosing regimens and/or reduced drug cost) will be apparent to those of skill in the art.

In yet another embodiment, provided is the use of a compound selected from the group consisting of:

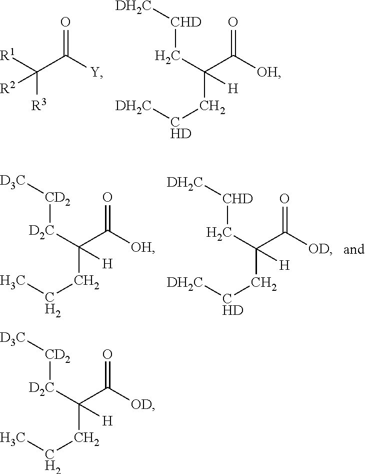

or a pharmaceutically acceptable salt thereof; where R¹, R², R³ and Y are as defined above, alone or together with one or more of the above-described, corresponding second therapeutic agents in the manufacture of a medicament, either as a single composition or as separate dosage forms, for treatment or prevention in a patient of a disease, disorder or symptom set forth above. Also provided is a compound of any of the above formulae or a composition comprising a compound of any of the above formulae for use in the treatment or prevention in a patient of a corresponding disease, disorder or symptom thereof delineated herein.

In one aspect, the compound of Formula I:

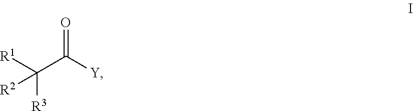

or a pharmaceutically acceptable salt thereof, wherein:

each of R¹ and R² independently for each occurrence is —CH₂CH₂CH₃ is optionally substituted with 1 to 7 deuterium atoms;

R³ is hydrogen or deuterium; and

Y is OH or OD,

provided that:

if each of R¹ and R² is —CH₂CH₂CH₃ or —CH₂CHD-CH₂D, then R³ is deuterium; or

if R¹ is —CH₂CH₂CH₃ and R² is —CD₂CD₂CD₃, then R³ is deuterium;

or a pharmaceutical composition comprising the compound of Formula I or salt thereof, is for use in treating a disease or condition selected from epilepsy, bipolar disorder, migraine, schizophrenia, autism, mood disorder, major depressive disorder, borderline personality disorder, intermittent explosive disorder, ADHD, dementia, Alzheimer's disease, amyotrophic lateral sclerosis, Huntington's disease, bronchial asthma, post-traumatic seizure disorder, spinal muscular atrophy, progressive supranuclear palsy (PSP), opiate dependence, drug dependence, substance withdrawal syndrome, nasopharyngeal carcinoma, acute myelogenous leukemia, myelodysplastic syndromes, lymphoma, advanced neoplasms, cervical cancer, breast cancer, malignant melanoma, Kaposi's sarcoma, lung cancer, neurologic cancer, basal cell carcinoma, squamous cell carcinoma, keratoacanthoma, Bowen disease, cutaneous T-cell lymphoma, non-small cell lung cancer, pre-malignant lesions, inflammations of the skin and/or mucosa, exposure to UV light, and sunburn. In a more specific aspect the compound or composition is for use in treating a disease or condition selected from epilepsy, bipolar disorder, migraine, schizophrenia, autism, mood disorder, major depressive disorder, borderline personality disorder, intermittent explosive disorder, ADHD, dementia, Alzheimer's disease, amyotrophic lateral sclerosis, Huntington's disease, bronchial asthma, post-traumatic seizure disorder, spinal muscular atrophy, progressive supranuclear palsy (PSP), opiate dependence, drug dependence, substance withdrawal syndrome, nasopharyngeal carcinoma, acute myelogenous leukemia, myelodysplastic syndromes, lymphoma, advanced neoplasms, cervical cancer, breast cancer, malignant melanoma, Kaposi's sarcoma, lung cancer, and neurologic cancer. In an even more specific aspect, the compound of composition is for use in treating a disease or condition selected from epilepsy, bipolar disorder, and migraine.

In another aspect, a compound selected from

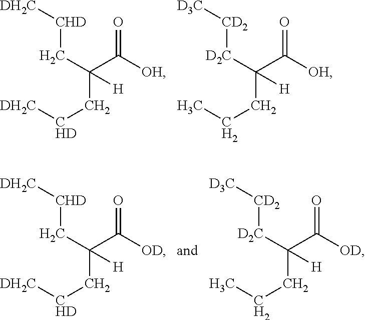

or a pharmaceutically acceptable salt thereof, or a composition comprising the compound or the salt is for use in treating a disease or condition selected from bipolar disorder, migraine, schizophrenia, autism, mood disorder, major depressive disorder, borderline personality disorder, intermittent explosive disorder, ADHD, dementia, Alzheimer's disease, amyotrophic lateral sclerosis, Huntington's disease, bronchial asthma, post-traumatic seizure disorder, spinal muscular atrophy, progressive supranuclear palsy (PSP), opiate dependence, drug dependence, substance withdrawal syndrome, nasopharyngeal carcinoma, acute myelogenous leukemia, myelodysplastic syndromes, lymphoma, advanced neoplasms, cervical cancer, breast cancer, malignant melanoma, Kaposi's sarcoma, lung cancer, neurologic cancer, basal cell carcinoma, squamous cell carcinoma, keratoacanthoma, Bowen disease, cutaneous T-cell lymphoma, non-small cell lung cancer, pre-malignant lesions, inflammations of the skin and/or mucosa, exposure to UV light, and sunburn. In a more specific aspect the compound or composition is for use in treating a disease or condition selected from epilepsy, bipolar disorder, migraine, schizophrenia, autism, mood disorder, major depressive disorder, borderline personality disorder, intermittent explosive disorder, ADHD, dementia, Alzheimer's disease, amyotrophic lateral sclerosis, Huntington's disease, bronchial asthma, post-traumatic seizure disorder, spinal muscular atrophy, progressive supranuclear palsy (PSP), opiate dependence, drug dependence, substance withdrawal syndrome, nasopharyngeal carcinoma, acute myelogenous leukemia, myelodysplastic syndromes, lymphoma, advanced neoplasms, cervical cancer, breast cancer, malignant melanoma, Kaposi's sarcoma, lung cancer, and neurologic cancer. In an even more specific aspect, the compound of composition is for use in treating a disease or condition selected from epilepsy, bipolar disorder, and migraine.

In another aspect, a compound of Formula I, or a compound selected from

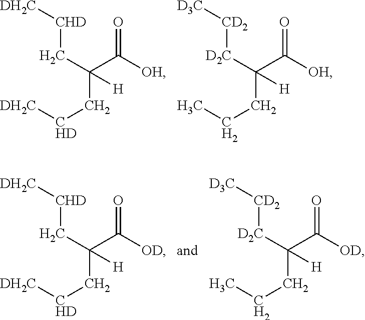

or a pharmaceutically acceptable salt or any of the foregoing, or a pharmaceutical composition comprising the compound or salt thereof is for use in treating cancer; and the compound or composition is used in conjunction with a second therapeutic agent selected from one of more of: sunitinib, sorafenib, dasatinib, erlotinib, lapatinib, lenalidomide, temozolomide, 5-fluorouracil, epirubicin, cyclophosphamide, karenitecin, and decitabine.

In another aspect, a compound of Formula I, or a compound selected from

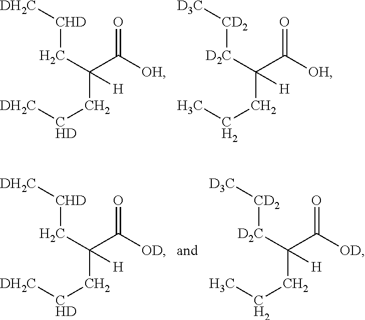

or a pharmaceutically acceptable salt or any of the foregoing, or a pharmaceutical composition comprising the compound or salt thereof is for use in treating bipolar disorder; and the compound or composition is used in conjunction with a second therapeutic agent selected from one of more of: aripiprazole, olanzapine, and lamotrigine.

In another aspect, the compound of Formula I, or a compound selected from

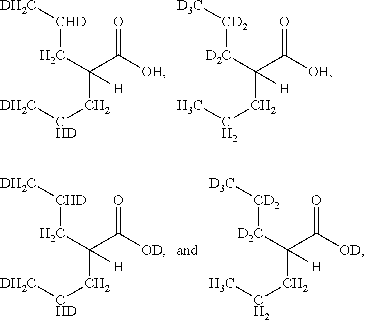

or a pharmaceutically acceptable salt or any of the foregoing, or a pharmaceutical composition comprising the compound or salt thereof is for use in treating dementia, and the compound or composition is used in conjunction with one or more of risperidone, quetiapine, and olanzapine.

In another aspect, a compound of Formula I, or a pharmaceutically acceptable salt thereof, or a pharmaceutical composition comprising the compound of Formula I or salt thereof is for use in treating epilepsy, and the compound or composition is used in conjunction with one or more of carbamazepine and phenyloin.

The term “used in conjunction with” as used herein means administered simultaneously with, or administered within 24 hours of the subject compound(s).

### Pharmaceutical Kits

The present disclosure also provides kits for use to treat bipolar disorder, migraine, schizophrenia, autism, mood disorder, major depressive disorder, borderline personality disorder, intermittent explosive disorder, ADHD, dementia, Alzheimer's disease, amyotrophic lateral sclerosis, Huntington's disease, bronchial asthma, post-traumatic seizure disorder, spinal muscular atrophy, progressive supranuclear palsy (PSP), opiate dependence, drug dependence; substance withdrawal syndrome, nasopharyngeal carcinoma, acute myelogenous leukemia, myelodysplastic syndromes, lymphoma, advanced neoplasms, cervical cancer, breast cancer, malignant melanoma, Kaposi's sarcoma, lung cancer, and neurologic cancer. These kits comprise (a) a pharmaceutical composition comprising a compound selected from the group consisting of:

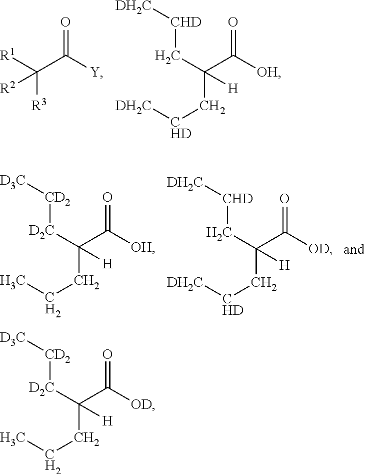

or a pharmaceutically acceptable salt thereof; where R¹, R², R³ and Y are as defined above; and (b) instructions describing a method of using the pharmaceutical composition to treat bipolar disorder, migraine, schizophrenia, autism, mood disorder, major depressive disorder, borderline personality disorder, intermittent explosive disorder, ADHD, dementia, Alzheimer's disease, amyotrophic lateral sclerosis, Huntington's disease, bronchial asthma, post-traumatic seizure disorder, spinal muscular atrophy, progressive supranuclear palsy (PSP), opiate dependence, drug dependence; substance withdrawal syndrome, nasopharyngeal carcinoma, acute myelogenous leukemia, myelodysplastic syndromes, lymphoma, advanced neoplasms, cervical cancer, breast cancer, malignant melanoma, Kaposi's sarcoma, lung cancer, and neurologic cancer.

In another embodiment, provided are kits for use to treat epilepsy. These kits comprise (a) a pharmaceutical composition comprising a compound of the Formula I:

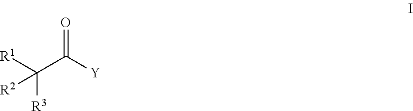

or a pharmaceutically acceptable salt thereof, where R¹, R², R³ and Y are as defined above; and (b) instructions describing a method of using the pharmaceutical composition to treat epilepsy.

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. In one embodiment, the container is a blister pack.

The kits of this disclosure may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

In an embodiment of the kits of this invention, the composition comprising the second active agent may be in a vessel or container that is separate from the vessel containing the composition comprising a compound of Formula I.

### Example 1

### Evaluation of Metabolic Stability

Microsomal Assay: Human liver microsomes (20 mg/mL) are obtained from Xenotech, LLC (Lenexa, Kans.). β-nicotinamide adenine dinucleotide phosphate, reduced form (NADPH), magnesium chloride (MgCl₂), and dimethyl sulfoxide (DMSO) are purchased from Sigma-Aldrich.

Determination of Metabolic Stability: 7.5 mM stock solutions of test compounds are prepared in DMSO. The 7.5 mM stock solutions are diluted to 12.5-50 μM in acetonitrile (ACN). The 20 mg/mL human liver microsomes are diluted to 0.625 mg/mL in 0.1 M potassium phosphate buffer, pH 7.4, containing 3 mM MgCl₂. The diluted microsomes are added to wells of a 96-well deep-well polypropylene plate in triplicate. A 10 μL aliquot of the 12.5-50 μM test compound is added to the microsomes and the mixture is pre-warmed for 10 minutes. Reactions are initiated by addition of pre-warmed NADPH solution. The final reaction volume is 0.5 mL and contains 0.5 mg/mL human liver microsomes, 0.25-1.0 μM test compound, and 2 mM NADPH in 0.1 M potassium phosphate buffer, pH 7.4, and 3 mM MgCl₂. The reaction mixtures are incubated at 37° C., and 50 μL aliquots are removed at 0, 5, 10, 20, and 30 minutes and added to shallow-well 96-well plates which contain 50 μL of ice-cold ACN with internal standard to stop the reactions. The plates are stored at 4° C. for 20 minutes after which 100 μL of water is added to the wells of the plate before centrifugation to pellet precipitated proteins. Supernatants are transferred to another 96-well plate and analyzed for amounts of parent remaining by LC-MS/MS using an Applied Bio-systems API 4000 mass spectrometer. The same procedure is followed for the non-deuterated counterpart of the compound of Formula I and the positive control, 7-ethoxycoumarin (1 μM). Testing is done in triplicate.

Data analysis: The in vitro t_{1/2}s for test compounds are calculated from the slopes of the linear regression of % parent remaining (ln) vs incubation time relationship.

in vitro t_{1/2}=0.693/k

k=−[slope of linear regression of % parent remaining(ln) vs incubation time]

Data analysis is performed using Microsoft Excel Software.

Without further description, it is believed that one of ordinary skill in the art can, using the preceding description and the illustrative examples, make and utilize the compounds of the present disclosure and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the disclosure. All the patents, journal articles and other documents discussed or cited above are herein incorporated by reference.

We claim:

1. A compound selected from the group consisting of: 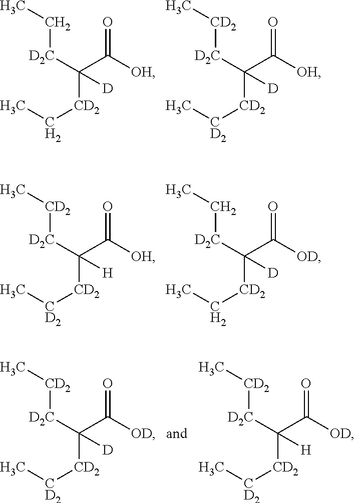
or a pharmaceutically acceptable salt of any of the foregoing.

2. A pharmaceutically acceptable composition comprising a compound of claim 1 or pharmaceutically acceptable salt thereof and a pharmaceutically acceptable carrier.

3. A pharmaceutically acceptable composition comprising:
a. a compound of the Formula I: 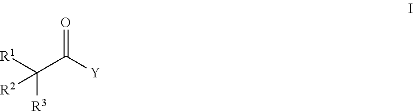
or a pharmaceutically acceptable salt thereof, wherein:
each of R¹ and R² independently for each occurrence is —CH₂CH₂CH₃, wherein —CH₂CH₂CH₃ is optionally substituted with 1 to 7 deuterium atoms;
R³ is hydrogen or deuterium; and
Y is OH or OD,
provided that if each of R¹ and R² is —CH₂CH₂CH₃ or —CH₂—CHD-CH₂D, then R³ is deuterium; further provided that if R¹ is —CH₂CH₂CH₃ and R² is —CD₂CD₂CD₃, then R³ is deuterium;
b. a second therapeutic agent selected from the group consisting of sunitinib, sorafenib, dasatinib, erlotinib, lapatinib, lenalidomide, temozolomide, 5-fluorouracil, epirubicin, cyclophosphamide, karenitecin, decitabine, aripiprazole, olanzapine, lamotrigine, risperidone, quetiapine, carbamazepine, and phenyloin; and
c. a pharmaceutically acceptable carrier.

4. A pharmaceutically acceptable composition comprising:
a. a compound of the formula: 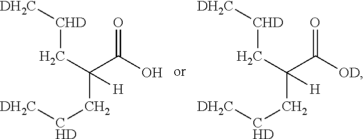
or a pharmaceutically acceptable salt thereof;
b. a second therapeutic agent selected from the group consisting of sunitinib, sorafenib, dasatinib, erlotinib, lapatinib, lenalidomide, temozolomide, 5-fluorouracil, epirubicin, cyclophosphamide, karenitecin, decitabine, aripiprazole, olanzapine, lamotrigine, risperidone, quetiapine, and phenyloin; and
c. a pharmaceutically acceptable carrier.

5. A pharmaceutically acceptable composition comprising:
a. a compound of the formula 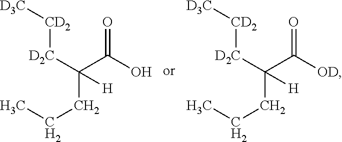
or a pharmaceutically acceptable salt thereof;
b. a second therapeutic agent selected from the group consisting of sunitinib, sorafenib, dasatinib, erlotinib, lapatinib, lenalidomide, temozolomide, 5-fluorouracil, epirubicin, cyclophosphamide, karenitecin, decitabine, aripiprazole, olanzapine, lamotrigine, risperidone, and quetiapine; and
c. a pharmaceutically acceptable carrier.

6. A compound of the Formula I: 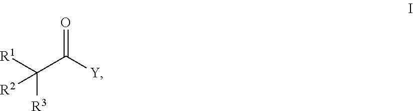
or a pharmaceutically acceptable salt thereof, wherein:
each of R¹ and R² independently for each occurrence is —CH₂CH₂CH₃, wherein —CH₂CH₂CH₃ is optionally substituted with 1 to 7 deuterium atoms;
R³ is hydrogen or deuterium; and
Y is OH or OD,
provided that if each of R¹ and R² is —CH₂CH₂CH₃ or —CH₂CHD-CH₂D, then R³ is deuterium; further provided that if R¹ is —CH₂CH₂CH₃ and R² is —CD₂CD₂CD₃, then R³ is deuterium; or a pharmaceutical composition comprising the compound or salt for use in treating a disease or condition selected from epilepsy, bipolar disorder, migraine, schizophrenia, autism, mood disorder, major depressive disorder, borderline personality disorder, intermittent explosive disorder, ADHD, dementia, Alzheimer's disease, amyotrophic lateral sclerosis, Huntington's disease, bronchial asthma, post-traumatic seizure disorder, spinal muscular atrophy, progressive supranuclear palsy (PSP), opiate dependence, drug dependence, substance withdrawal syndrome, nasopharyngeal carcinoma, acute myelogenous leukemia, myelodysplastic syndromes, lymphoma, advanced neoplasms, cervical cancer, breast cancer, malignant melanoma, Kaposi's sarcoma, lung cancer, neurologic cancer, basal cell carcinoma, squamous cell carcinoma, keratoacanthoma, Bowen disease, cutaneous T-cell lymphoma, non-small cell lung cancer, pre-malignant lesions, inflammations of the skin and/or mucosa, exposure to UV light, and sunburn.

7. The compound or composition of claim 6, for use in treating a disease or condition selected from epilepsy, bipolar disorder, migraine, schizophrenia, autism, mood disorder, major depressive disorder, borderline personality disorder, intermittent explosive disorder, ADHD, dementia, Alzheimer's disease, amyotrophic lateral sclerosis, Huntington's disease, bronchial asthma, post-traumatic seizure disorder, spinal muscular atrophy, progressive supranuclear palsy (PSP), opiate dependence, drug dependence, substance withdrawal syndrome, nasopharyngeal carcinoma, acute myelogenous leukemia, myelodysplastic syndromes, lymphoma, advanced neoplasms, cervical cancer, breast cancer, malignant melanoma, Kaposi's sarcoma, lung cancer, and neurologic cancer.

8. The compound or composition of claim 7, for use in treating a disease or condition selected from epilepsy, bipolar disorder, and migraine.

9. The compound or composition of any one of claims 6 to 8, wherein the compound is selected from the group consisting of: 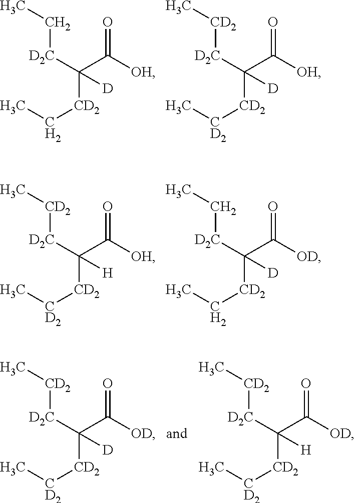
or a pharmaceutically acceptable salt thereof.

10. A compound selected from: 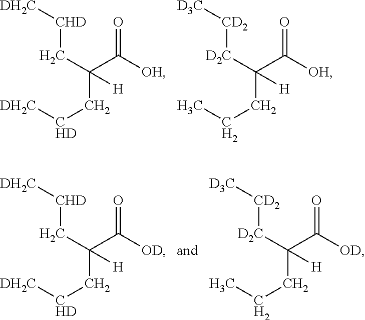
or a pharmaceutically acceptable salt thereof, or a composition comprising the compound or salt for use in treating a disease or condition selected from the group consisting of bipolar disorder, migraine, schizophrenia, autism, mood disorder, major depressive disorder, borderline personality disorder, intermittent explosive disorder, ADHD, dementia, Alzheimer's disease, amyotrophic lateral sclerosis, Huntington's disease, bronchial asthma, post-traumatic seizure disorder, spinal muscular atrophy, progressive supranuclear palsy (PSP), opiate dependence, drug dependence, substance withdrawal syndrome, nasopharyngeal carcinoma, acute myelogenous leukemia, myelodysplastic syndromes, lymphoma, advanced neoplasms, cervical cancer, breast cancer, malignant melanoma, Kaposi's sarcoma, lung cancer, neurologic cancer, basal cell carcinoma, squamous cell carcinoma, keratoacanthoma, Bowen disease, cutaneous T-cell lymphoma, non-small cell lung cancer, pre-malignant lesions, inflammations of the skin and/or mucosa, exposure to UV light, and sunburn.

11. The compound or composition of claim 10, for use in treating a disease or condition selected from the group consisting of bipolar disorder, migraine, schizophrenia, autism, mood disorder, major depressive disorder, borderline personality disorder, intermittent explosive disorder, ADHD, dementia, Alzheimer's disease, amyotrophic lateral sclerosis, Huntington's disease, bronchial asthma, post-traumatic seizure disorder, spinal muscular atrophy, progressive supranuclear palsy (PSP), opiate dependence, drug dependence, substance withdrawal syndrome, nasopharyngeal carcinoma, acute myelogenous leukemia, myelodysplastic syndromes, lymphoma, advanced neoplasms, cervical cancer, breast cancer, malignant melanoma, Kaposi's sarcoma, lung cancer, and neurologic cancer.

12. The compound or composition of claim 11, for use in treating a disease or condition selected from bipolar disorder and migraine.

13. The compound or composition of any one of claims 6 to 12 for use in treating cancer, wherein the compound of composition is used in conjunction with a second therapeutic agent selected from one of more of: sunitinib, sorafenib, dasatinib, erlotinib, lapatinib, lenalidomide, temozolomide, 5-fluorouracil, epirubicin, cyclophosphamide, karenitecin, and decitabine.

14. The compound or composition of any one of claims 6 to 12 for use in treating bipolar disorder wherein the compound of composition is used in conjunction with a second therapeutic agent selected from one of more of: aripiprazole, olanzapine, and lamotrigine.

15. The compound or composition of any one of claims 6 to 12 for use in treating dementia wherein the compound of composition is used in conjunction with a second therapeutic agent selected from one or more of risperidone, quetiapine, and olanzapine.

16. The compound or composition of any one of claims 6 to 9 for use in treating epilepsy wherein the compound of composition is used in conjunction with a second therapeutic agent selected from one or more of carbamazepine and phenyloin.
